(19) United States
(12) Patent Application Publication
(10) Pub. No.: US 2022/0054268 A1
(43) Pub. Date: Feb. 24, 2022

(54) RING CONTRACTION DEVICE AND MOUNTING METHOD THEREOF

(71) Applicant: SHANGHAI HONGYU MEDICAL TECHNOLOGY CO. LTD, Shanghai (CN)

(72) Inventors: Yufeng DAI, Shanghai (CN); Bingyue PAN, Shanghai (CN); Lingchuan ZHU, Shanghai (CN)

(21) Appl. No.: 17/478,713

(22) Filed: Sep. 17, 2021

Related U.S. Application Data
(63) Continuation of application No. PCT/CN2021/098100, filed on Jun. 3, 2021.

(30) Foreign Application Priority Data
Aug. 18, 2020 (CN) 202010832122.2

(51) Int. Cl. A61F 2/24

(52) U.S. Cl. CPC A61F 2/2454; A61F 2/246; A61F 2220/0016; A61F 2220/0075; A61F 2230/0093; A61F 2250/0065; A61F 2250/0007; A61F 2210/0014

(57) ABSTRACT

The present invention discloses a ring contraction device and a mounting method thereof, and relates to the field of medical instruments. The ring contraction device includes a plugging piece, a hemostatic disc, a puncture tube and a push rod. One end of the push rod penetrates through an inner cavity of the puncture tube and extends to the outside of the inner cavity of the puncture tube. The push rod is configured to push out the plugging piece in the puncture tube to be propped open. The hemostatic disc is further arranged at a pull wire to be connected to the plugging piece. A distance between the plugging piece and the hemostatic disc can be effectively adjusted through the pull wire, so as to achieve a ring contraction effect. The ring contraction device has a reasonable structure.

### CROSS-REFERENCE TO RELATED APPLICATIONS

This application is a bypass continuation application of PCT application no.: PCT/CN2021/098100. This application claims priorities from PCT Application No. PCT/CN2021/098100, filed Jun. 3, 2021, and from the Chinese patent application 202010832122.2 filed Aug. 18, 2020, the contents of which are incorporated herein in the entirety by reference.

### TECHNICAL FIELD

The present invention relates to the field of medical devices, and more particularly, to a ring contraction device used as an aid in a mitral valve surgery and a mounting method thereof.

### BACKGROUND

A mitral valve, i.e., a left atrioventricular valve, also referred to as “bicuspid valve”, is attached to a left fibrous atrioventricular ring and is formed by folds of endocardia. It has two valves, the anteromedial one is an anterior cusp, which is larger and often called a macrovalve, and marks a boundary between inflow and outflow tracts of a left ventricle, and the posterolateral one is a posterior cusp, which is smaller and often called a microvalve. The valve is triangular and pointed towards a left ventricular cavity. Bottom margins of the two valves often fuse with each other, and sometimes small accessory valves appear between the two valves. Many chordae tendineae attach to a papillary muscle on valve cusps, margins and ventricular surfaces. When the ventricles contract, the mitral valve tightly closes an atrioventricular opening to prevent the flow of blood back into a left atrium. Therefore, the treatment of mitral valve regurgitation requires the use of a ring contraction device.

The treatment of mitral valve regurgitation requires the use of the ring contraction device; however, a conventional ring contraction device has a simple structure, single function and complex operation. Moreover, the conventional ring contraction device is mostly of a rigid structure, which increases the difficulty of operation and prolongs the operation time, thereby causing unnecessary harm to patients. Secondly, the conventional ring contraction device is not provided with a puncture needle device, which makes the treatment of mitral valve regurgitation more traumatic. Therefore, an elastic or flexible ring contraction device with a simple structure and a puncture structure is urgently needed.

### SUMMARY

An objective of the present invention is to provide a ring contraction device and a mounting method thereof, so as to solve the above problem.

In order to achieve the above objective, the present invention provides the following technical solution. A ring contraction device, including a plugging piece, a puncture tube, a push rod, a column, a hemostatic disc, a first pull wire and a second pull wire, wherein the plugging piece has a shape memory function and is pressed into a sheath tube of a smaller size, the plugging piece is fixed by a fixing ring, the fixing ring protrudes from a top end of the puncture tube during operation, and the fixing ring is communicated with the plugging piece to be compressed into the puncture tube when in a non-operation state; a lower end of the puncture tube is coaxially sleeved over an upper end of the push rod adjustably, a lower end of the push rod is fixedly connected to an upper end of the column, two through holes are arranged parallel to axes of the push rod and the column, the first pull wire is arranged in one through hole to be connected to one side of the fixing ring, and the second pull wire is arranged in the other through hole to be connected to the other side of the fixing ring; and the plugging piece is connected to the hemostatic disc by a pull wire.

Preferably, two perforations are provided in the hemostatic disc to facilitate the passage of the first pull wire and the second pull wire, so as to play a role of ring contraction by adjusting a radial length of the hemostatic disc and adjusting a distance between the plugging piece and the hemostatic disc through the pull wire.

Preferably, an adjusting sleeve is arranged at a sleeving end of the puncture tube and the push rod, the puncture tube is fixedly sleeved with the adjusting sleeve, and the push rod is slidably sleeved with the puncture tube; two tube walls radially passing through a same side on the adjusting sleeve and the puncture tube are provided with threaded holes, an adjusting handle is screwed into the threaded hole, and the push rod is positioned in the puncture tube by the adjusting handle.

Preferably, one end of the puncture tube is needle-like.

Preferably, the plugging piece of the ring contraction device is made of an elastic nickel-titanium alloy material; the fixing ring is composed of a shape memory metal or a shape memory polymer material with a shape memory function.

Preferably, the plugging piece is a shrinkable and expandable wire woven mesh and is connected at least partially to the fixing ring, is cylindrically received in the puncture tube in a contraction state, and expands into a woven mesh in a shape of a grid umbrella canopy after being pushed out of the puncture tube by the push rod.

Preferably, the plugging piece is an elastic telescopic disc, the elastic telescopic disc has openings on two opposite sides, in the middle and at a bottom, the first pull wire and the second pull wire pass through the bottom opening to the openings on the two sides, plugs are arranged at the openings on the two sides, a diameter of the plug is greater than those of two end openings, and the plug is connected to the first pull wire and the second pull wire.

Preferably, when a transverse pipe is propped open, it is concave from one side opposite to the middle opening to form an elastic adjustable arc structure.

Preferably, the pull wire is made of a polymer material and has a smooth surface.

Preferably, the hemostatic disc is of a flat plate or sheet structure and may be in a shape of a disk, a star, a square, or other irregularly shaped structures. The hemostatic disc is made of a pet material, a medical silica gel, a peek material, or a composite material of at least two or more materials.

Operation principle is as follows. Firstly, the plugging piece is compressed into a straight line state to be received in a puncture tube hole, an atrial septum in the left atrium near a position P2 is punctured through the puncture tube, and the plugging piece is pushed through the push rod to open the plugging piece into a web disk; then, the puncture tube and the push rod are exited in turn, after the withdrawing, the first pull wire and the second pull wire are inserted into the hole of the hemostatic disc and then the hemostatic disc is attached to the surface of the heart for hemostasis; an atrium and a valve ring are shrunk by cross-knotting the first pull wire and the second pull wire to reduce a regurgitant surface. The ring contraction device has a simple structure, complete function and simple operation. The plugging piece is made of a flexible material, which has less harm to the tissue and easily makes the tissue grow and recover, so as to achieve a minimally invasive purpose.

The present invention has the following technical effects and advantages.

1. The present invention has a reasonable structure. The plugging piece is compressed into a straight line state to be received in a puncture tube hole, an atrial septum in the left atrium near a position P (puncture position) is punctured through the puncture tube, and the plugging piece is pushed through the push rod to open the plugging piece into a web disk; then, the puncture tube and the push rod are exited in turn, after the withdrawing, the first pull wire and the second pull wire are inserted into the hole of the hemostatic disc and then the hemostatic disc is attached to the surface of the heart for hemostasis; an atrium and a valve ring are shrunk by cross-knotting the first pull wire and the second pull wire to reduce a regurgitant surface.

2. In the present invention, by means of the flexible design of the plugging piece, operative wounds are small, and tissue recovery is fast, so as to achieve a purpose of treating reflux. The pull wire is made of high polymer materials and thus has a smooth surface, which can achieve a minimally invasive purpose. Devices are relatively small and are all delivered by smooth round tubes, the path is relatively simple, an operation is easy to operate, and production, research and development are easy to achieve, thereby reducing operation costs.

### BRIEF DESCRIPTION OF THE DIAGRAMS

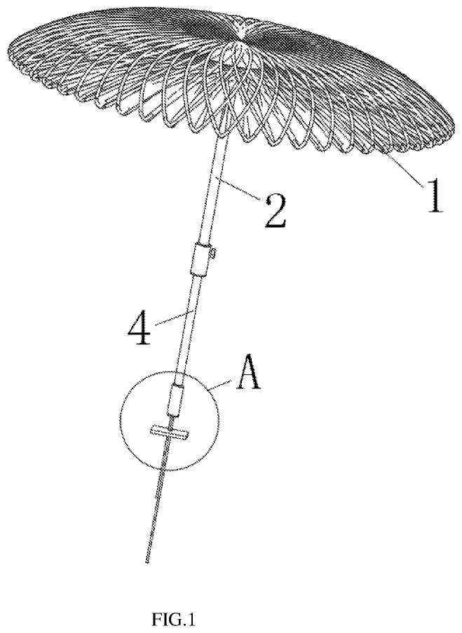

FIG. 1 is a schematic diagram of a three-dimensional structure of the present invention;

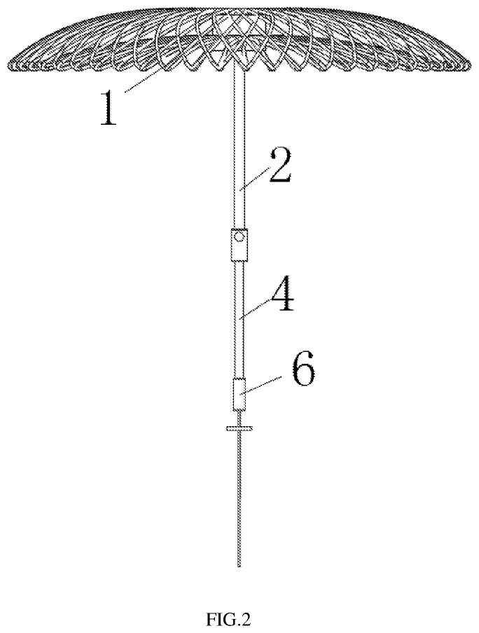

FIG. 2 is a schematic diagram of a front structure of the present invention;

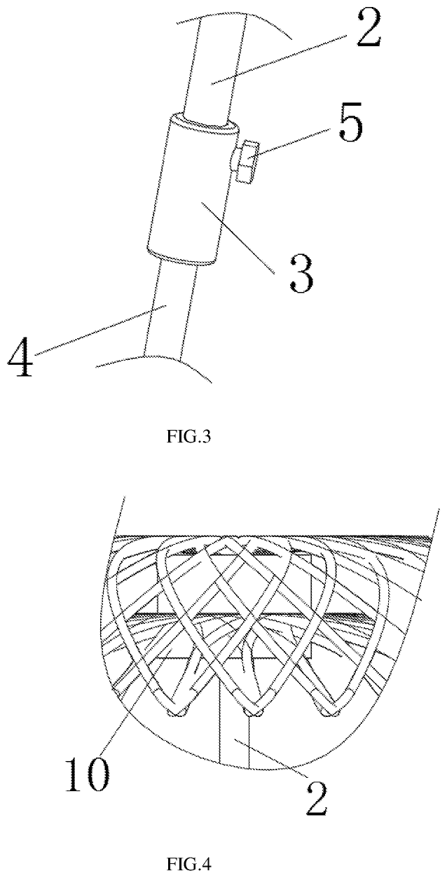

FIG. 3 is a schematic diagram of a partial enlarged structure of the present invention;

FIG. 4 is a schematic diagram of a partial enlarged structure of the present invention;

FIG. 5 is a schematic diagram of an enlarged structure at A in FIG. 1 according to the present invention;

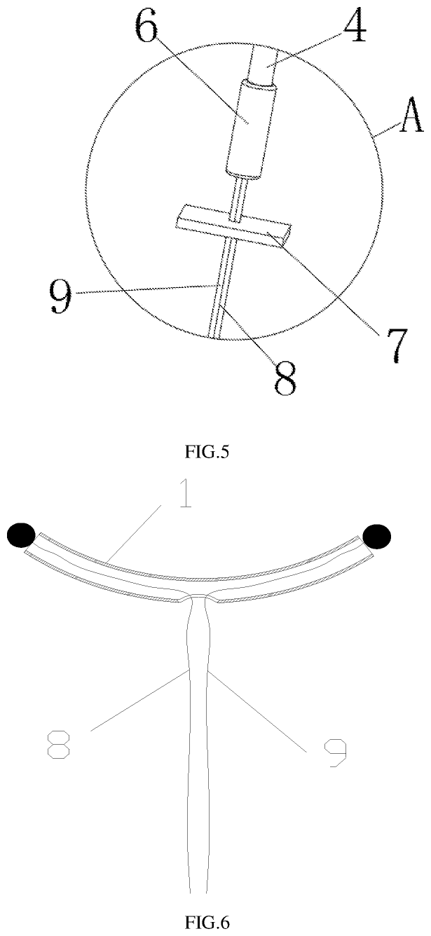

FIG. 6 is a schematic diagram of a second embodiment of a plugging device;

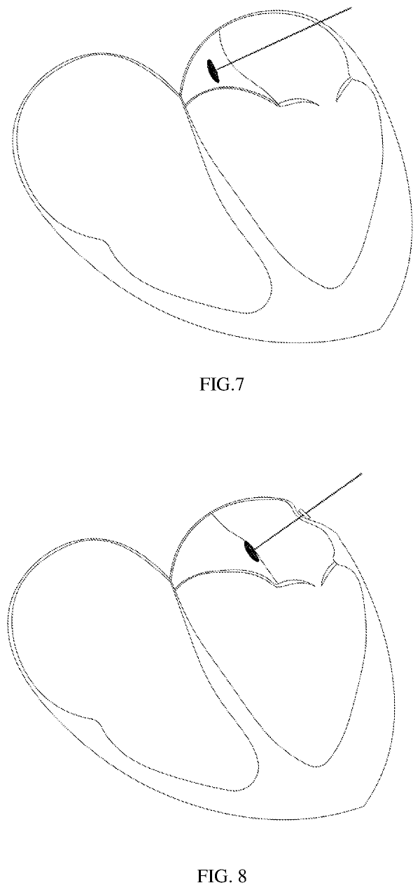

FIG. 7 is a schematic diagram of a device implantation path according to the present invention; and

FIG. 8 is a schematic diagram after device implantation according to the present invention.

In the drawings, 1: plugging device; 2: puncture tube; 3: adjusting sleeve; 4: push rod; 5: locking screw; 6: column; 7: hemostatic disc; 8: first pull wire; 9: second pull wire; 10: fixing ring.

### DETAILED DESCRIPTION OF EMBODIMENTS

The technical solutions in the embodiments of the present invention will be described clearly and completely below with reference to the accompanying drawings in the embodiments of the present invention. The described embodiments are merely some of rather than all of the embodiments of the present invention. All other embodiments acquired by those of ordinary skill in the art without creative efforts based on the embodiments of the present invention shall fall within the protection scope of the present invention.

The present invention provides a ring contraction device applied to mitral valves as shown in FIG. 1 to FIG. 6. The ring contraction device includes a plugging device 1, a puncture tube 2, a push rod 4, a column 6, a hemostatic disc 7, a first pull wire 8, a second pull wire 9 and a fixing ring 10.

Connection relationship is as follows. The plugging device 1 has a shape memory function and can be pressed into a sheath tube of a smaller size. The plugging piece 1 is fixed by the fixing ring 10, and the fixing ring 10 also has a shape memory function. The fixing ring 10 protrudes from a top end of the puncture tube 2 during operation, and the fixing ring 10 is communicated with the plugging piece 1 to be compressed into the puncture tube 2 when in a non-operation state. A lower end of the puncture tube 2 is coaxially sleeved over an upper end of the push rod 4 adjustably, a lower end of the push rod 4 is fixedly connected to an upper end of the column 6, two through holes are arranged parallel to axes of the push rod 4 and the column 6, the first pull wire 8 is arranged in one through hole to be connected to one side of the fixing ring 10, and the second pull wire 9 is arranged in the other through hole to be connected to the other side of the fixing ring 10. The plugging piece 1 is connected to the hemostatic disc 7 by a pull wire.

Two perforations are provided in the hemostatic disc 7 to facilitate the passage of the first pull wire 8 and the second pull wire 9, so as to play a role of ring contraction by adjusting a radial length of the hemostatic disc 7 and adjusting a distance between the plugging piece 1 and the hemostatic disc 7 through the pull wire.

Further, an adjusting sleeve 3 is arranged at a sleeving end of the puncture tube 2 and the push rod 4, the puncture tube 2 is fixedly sleeved with the adjusting sleeve 3, and the push rod 4 is slidably sleeved with the puncture tube 2; two tube walls radially passing through a same side on the adjusting sleeve 3 and the puncture tube 2 are provided with threaded holes, an adjusting handle 5 is screwed into the threaded hole, and the push rod 4 is positioned in the puncture tube 2 by the adjusting handle 5.

Further, the two perforations are provided in the hemostatic disc 7 to facilitate the passage of the first pull wire 8 and the second pull wire 9. The hemostatic disc 7 is of a flat plate or sheet structure, and is made of a pet material, a medical silica gel, a peek material, or a composite material of at least two or more materials.

One end of the puncture tube 2 is needle-like, so as to facilitate the puncturing.

Further, the plugging piece 1 is a shrinkable and expandable wire woven mesh and is connected at least partially to the fixing ring 10, is cylindrically received in the puncture tube 2 in a contraction state, and expands into a woven mesh in a shape of a grid umbrella canopy after being pushed out of the puncture tube 2 by the push rod 4.

In another embodiment, the plugging piece 1 is an elastic telescopic disc, the elastic telescopic disc has openings on two opposite sides, in the middle and at a bottom, the first pull wire 8 and the second pull wire 9 pass through the bottom opening to the openings on the two sides, plugs are arranged at the openings on the two sides, a diameter of the plug is greater than those of two end openings, and the plug is connected to the first pull wire 8 and the second pull wire 9.

One side of the adjusting handle 5 is knurled.

Material: the plugging piece 1 is made of an elastic nickel-titanium alloy material; the fixing ring 10 is composed of a shape memory metal or a shape memory polymer material with a shape memory function.

Referring to FIG. 7 and FIG. 8, a mounting method for the above ring contraction device includes:

S1: puncturing, preparing the ring contraction device, and puncturing an adjoining atrium with the puncture tube 2 receiving the plugging piece 1 in the contraction state;

S2: propping the plugging piece open, loosening the adjusting handle 5, pushing the push rod 4 by hand, pushing the plugging piece 1 out of the puncture tube 2, and opening the plugging piece 1 in an expanded state;

S3: withdrawing auxiliary parts, and after the plugging piece 1 is propped open, withdrawing the push rod 4 and the puncture tube 2 in turn; and

S4: mounting the hemostatic disc, passing the first pull wire 8 and the second pull wire 9 through two holes of the hemostatic disc 7 to be pushed against an exterior of a heart, and knotting the first pull wire 8 and the second pull wire 9 outside to shrink an atrium and a valve ring to reduce a regurgitant surface, so as to complete the mounting of the ring contraction device.

Finally, it should be noted that, the above embodiments are merely preferred embodiments of the present invention, but not to limit the present invention. Although the present invention is described in detail with reference to the foregoing embodiments, those skilled in the art may still make modifications to the technical solutions described in the foregoing embodiments or make equivalent replacements to some technical features thereof. Any modification, equivalent replacement, improvement and the like made within the spirit and scope of the present invention should all fall within the protection scope of the present invention.

1. A ring contraction device, comprising a plugging piece (1), a puncture tube (2), a push rod (4), a column (6), a hemostatic disc (7), a first pull wire (8) and a second pull wire (9), wherein the plugging piece (1) has a shape memory function and is pressed into a sheath tube of a smaller size, the plugging piece (1) is fixed by a fixing ring (10), the fixing ring (10) protrudes from a top end of the puncture tube (2) during operation, and the fixing ring (10) is communicated with the plugging piece (1) to be compressed into the puncture tube (2) when in a non-operation state; a lower end of the puncture tube (2) is coaxially sleeved over an upper end of the push rod (4) adjustably, a lower end of the push rod (4) is fixedly connected to an upper end of the column (6), two through holes are arranged parallel to axes of the push rod (4) and the column (6), the first pull wire (8) is arranged in one through hole to be connected to one side of the fixing ring (10), and the second pull wire (9) is arranged in the other through hole to be connected to the other side of the fixing ring (10); and the plugging piece (1) is connected to the hemostatic disc (7) by a pull wire.

2. The ring contraction device according to claim 1, wherein an adjusting sleeve (3) is arranged at a sleeving end of the puncture tube (2) and the push rod (4), the puncture tube (2) is fixedly sleeved with the adjusting sleeve (3), and the push rod (4) is slidably sleeved with the puncture tube (2); two tube walls radially passing through a same side on the adjusting sleeve (3) and the puncture tube (2) are provided with threaded holes, an adjusting handle (5) is screwed into the threaded hole, and the push rod (4) is positioned in the puncture tube (2) by the adjusting handle (5).

3. The ring contraction device according to claim 1, wherein two perforations are provided in the hemostatic disc (7) to facilitate the passage of the first pull wire (8) and the second pull wire (9), so as to play a role of ring contraction by adjusting a radial length of the hemostatic disc (7) and adjusting a distance between the plugging piece (1) and the hemostatic disc (7) through the pull wire.

4. The ring contraction device according to claim 1, wherein one end of the puncture tube (2) is needle-like.

5. The ring contraction device according to claim 1, wherein the plugging piece (1) is a shrinkable and expandable wire woven mesh and is connected at least partially to the fixing ring (10), is cylindrically received in the puncture tube (2) in a contraction state, and expands into a woven mesh in a shape of a grid umbrella canopy after being pushed out of the puncture tube (2) by the push rod (4).

6. The ring contraction device according to claim 1, wherein the plugging piece (1) is an elastic telescopic disc, the elastic telescopic disc has openings on two opposite sides, in the middle and at a bottom, the first pull wire (8) and the second pull wire (9) pass through the bottom opening to the openings on the two sides, plugs are arranged at the openings on the two sides, a diameter of the plug is greater than those of two end openings, and the plug is connected to the first pull wire (8) and the second pull wire (9).

7. The ring contraction device according to claim 5, wherein the plugging piece (1) of the ring contraction device is made of an elastic nickel-titanium alloy material; the fixing ring (10) is composed of a shape memory metal or a shape memory polymer material with a shape memory function.

8. The ring contraction device according to claim 2, wherein one side of the adjusting handle (5) is knurled.

9. The ring contraction device according to claim 3, wherein the hemostatic disc (7) is of a flat plate or sheet structure, and is made of a pet material, a medical silica gel, a peek material, or a composite material of at least two or more materials.

10. A ring contraction device mounting method for mitral valve surgeries by using the ring contraction device according to claim 1, wherein the method comprises:
S1: puncturing, preparing the ring contraction device, and puncturing an adjoining atrium with the puncture tube (2) receiving the plugging piece (1) in the contraction state;
S2: propping the plugging piece open, loosening the adjusting handle (5), pushing the push rod (4) by hand, pushing the plugging piece (1) out of the puncture tube (2), and opening the plugging piece (1) in an expanded state;
S3: withdrawing auxiliary parts, and after the plugging piece (1) is propped open, withdrawing the push rod (4) and the puncture tube (2) in turn; and
S4: mounting the hemostatic disc, passing the first pull wire (8) and the second pull wire (9) through two holes of the hemostatic disc (7) to be pushed against an exterior of a heart, and knotting the first pull wire (8) and the second pull wire (9) outside to shrink an atrium and a valve ring to reduce a regurgitant surface, so as to complete the mounting of the ring contraction device.

11. The ring contraction device according to claim 2, wherein two perforations are provided in the hemostatic disc (7) to facilitate the passage of the first pull wire (8) and the second pull wire (9), so as to play a role of ring contraction by adjusting a radial length of the hemostatic disc (7) and adjusting a distance between the plugging piece (1) and the hemostatic disc (7) through the pull wire.

12. The ring contraction device according to claim 6, wherein the plugging piece (1) of the ring contraction device is made of an elastic nickel-titanium alloy material; the fixing ring (10) is composed of a shape memory metal or a shape memory polymer material with a shape memory function.

13. The ring contraction device mounting method for mitral valve surgeries by using the ring contraction device according to claim 10, wherein an adjusting sleeve (3) is arranged at a sleeving end of the puncture tube (2) and the push rod (4), the puncture tube (2) is fixedly sleeved with the adjusting sleeve (3), and the push rod (4) is slidably sleeved with the puncture tube (2); two tube walls radially passing through a same side on the adjusting sleeve (3) and the puncture tube (2) are provided with threaded holes, an adjusting handle (5) is screwed into the threaded hole, and the push rod (4) is positioned in the puncture tube (2) by the adjusting handle (5).

14. The ring contraction device mounting method for mitral valve surgeries by using the ring contraction device according to claim 10, wherein two perforations are provided in the hemostatic disc (7) to facilitate the passage of the first pull wire (8) and the second pull wire (9), so as to play a role of ring contraction by adjusting a radial length of the hemostatic disc (7) and adjusting a distance between the plugging piece (1) and the hemostatic disc (7) through the pull wire.

15. The ring contraction device mounting method for mitral valve surgeries by using the ring contraction device according to claim 10, wherein one end of the puncture tube (2) is needle-like.

16. The ring contraction device mounting method for mitral valve surgeries by using the ring contraction device according to claim 10, wherein the plugging piece (1) is a shrinkable and expandable wire woven mesh and is connected at least partially to the fixing ring (10), is cylindrically received in the puncture tube (2) in a contraction state, and expands into a woven mesh in a shape of a grid umbrella canopy after being pushed out of the puncture tube (2) by the push rod (4).

17. The ring contraction device mounting method for mitral valve surgeries by using the ring contraction device according to claim 10, wherein the plugging piece (1) is an elastic telescopic disc, the elastic telescopic disc has openings on two opposite sides, in the middle and at a bottom, the first pull wire (8) and the second pull wire (9) pass through the bottom opening to the openings on the two sides, plugs are arranged at the openings on the two sides, a diameter of the plug is greater than those of two end openings, and the plug is connected to the first pull wire (8) and the second pull wire (9).

18. The ring contraction device mounting method for mitral valve surgeries by using the ring contraction device according to claim 10, wherein the plugging piece (1) of the ring contraction device is made of an elastic nickel-titanium alloy material; the fixing ring (10) is composed of a shape memory metal or a shape memory polymer material with a shape memory function.

19. The ring contraction device mounting method for mitral valve surgeries by using the ring contraction device according to claim 10, wherein one side of the adjusting handle (5) is knurled.

20. The ring contraction device mounting method for mitral valve surgeries by using the ring contraction device according to claim 10, wherein the hemostatic disc (7) is of a flat plate or sheet structure, and is made of a pet material, a medical silica gel, a peek material, or a composite material of at least two or more materials.
